(19) United States
(12) Patent Application Publication
(10) Pub. No.: US 2010/0310526 A1
(43) Pub. Date: Dec. 9, 2010

(54) COSMETIC METHOD FOR INCREASING THE PIGMENTATION OF SKIN USING MELANOCYTE PRECURSOR CELLS

(75) Inventor: Thomas Hunziker, Oberhofen (CH)

Correspondence Address: GREENBLUM & BERNSTEIN, P.L.C., 1950 ROLAND CLARKE PLACE, RESTON, VA, 20191, US

(73) Assignee: EURODERM GMBH, Leipzig (DE)

(21) Appl. No.: 12/682,988

(22) PCT Filed: Sep. 15, 2008
(86) PCT No.: PCT/EP08/07684

§ 371 Date: Aug. 25, 2010

(30) Foreign Application Priority Data
Oct. 15, 2007 (DE) 10 2007 049 402.7

(51) Int. Cl. A61K 8/98; A61Q 19/00; C12N 5/02

(52) U.S. Cl. 424/93.7; 435/378

(57) ABSTRACT

The present invention proposes a cosmetic method for increasing the pigmentation of skin with the steps application of melanocyte precursor cells from root sheaths collected from a first area of skin of a donor to a second area of skin of a recipient. Furthermore, the use of melanocyte precursor cells for increasing the pigmentation of skin as well as a method for producing a suspension having precursor cells from the root sheath is proposed.

The present invention relates to a method for increasing the pigmentation of skin according to the preamble of claim 1. It further relates to the use of melanocyte precursor cells according to claim 7 and a method for producing a suspension according to claim 11

People are known from practice whose skin has a reduced pigmentation at one or more points as measured by other cutaneous areas. This reduced pigmentation can appear more or less pale compared to skin with regular pigmentation and is perceived by some of those affected as esthetically or cosmetically objectionable. In the age of face lifts, the use of Botox to hide wrinkles and suctioning fat for cosmetic reasons, there is therefore a desire to also be able to bring about an increase or an intensification in the pigmentation of certain areas of the skin.

The object of the invention is therefore to disclose a suitable method for increasing or intensifying the pigmentation of skin as well as a method for producing a cell solution suitable hereby as well as application modalities for the same. The object according to the invention is attained through a method with the features of claim 1, through the use of melanocyte precursor cells according to claim 7 as well as through a method according to claim 11.

According to claim 1 there is proposed a method for increasing the pigmentation of the skin, which method comprises an application of melanocyte precursor cells from root sheaths of a first area of skin of a donor onto a second area of skin of a recipient.

The method according to the invention serves the cosmetic purpose of increasing the pigmentation of the skin of the second area of skin, thus fulfilling esthetic requirements of the people treated with the procedure. The method according to the invention of claim 1 therefore serves a non-therapeutic purpose.

The application of melanocyte precursor cells or melanocyte stem cells from root sheaths, in particular from epithelial root sheaths, of a first area of skin onto a second area of skin serves to increase the pigmentation of the second area of skin through pigment formation on the second area of skin by the formation in particular of melanocytes from the precursor cells with their known contribution to the pigmentation of the skin. The pigmentation can thereby be changed according to the external circumstances—such as, for example, the strength or the duration of an exposure to sunlight—and thus naturally adapts in a cosmetically advantageous manner to the pigmentation of the cutaneous area surrounding the second area of skin.

In contrast to the application of, for example, interfollicular melanocytes, the application of melanocyte precursor cells furthermore brings the advantage of a comparatively unlimited availability. While obtaining melanocytes as a rule is associated with the removal of skin and thus is a possibility only for increasing the pigmentation of a limited cutaneous area—and furthermore is associated with a surgical intervention into the integrity of the skin at the removal site—melanocyte precursor cells can be obtained from root sheaths relatively easily and in a high number. The application of melanocyte precursor cells thus implies the advantageously high availability thereof.

The application can be carried out, for example, by means of a suspension with 10²-10⁹, in particular with 10⁵-10⁷ cells/ml, for example, by means of a syringe or spray or in a biocompatible nonwoven.

This can be carried out in a biocompatible solution (e.g., PBS, fibrin) or by means of a biocompatible carrier (e.g., hyaluronan, collagen). The cells can thereby be employed as vital or growth arrested (e.g., growth arrested by means of mitomycin C or radiation) cells or also as cell extracts (such as, e.g., lyophilisates, sonicates). Conditioned media from these cells can also be used for this purpose.

The application of melanocyte precursor cells furthermore implies the advantageously simple collection thereof which can thus be carried out repeatedly. Thus the process of collecting these melanocyte precursor cells before their application can be carried out relatively simply and painlessly. Furthermore, there is no risk of complications associated with this process. Thus the hair used for obtaining the cells can be obtained, for example, by plucking hairs from the head, in particular anagen hair, in particular from the capillitium, which represents another advantage compared to the use of cells of other origin, in particular interfollicular melanocytes.

The melanocyte precursor cells can thereby come from a donor and then also be applied to the same again. The first area of skin and the second area of skin can thus be areas of skin of one and the same living being. However, the melanocyte precursor cells can also come from a donor and be applied to a recipient differing therefrom. It is unimportant thereby whether the donor and recipient are human or animal. A transfer from animal to human and vice versa is also encompassed by the invention. “Melanocyte precursor cells” for the purposes of the invention means both autologous as well as allogenic and xenogenic melanocyte precursor cells or melanocyte stem cells.

The application of the precursor cells can hereby be carried out by means of simple application onto the skin. The cells can thereby be fixed to the second area of the skin, e.g., by means of fibrin glue and protected with an occlusive dressing. However, any other suitable form of application, e.g., by integration of the cells into biological or synthetic matrices, is also possible according to the invention.

Advantageous further developments of the method according to the invention are thereby respectively the subject matter of the dependent claims.

In a preferred embodiment, the method according to the invention therefore has the collection of the melanocyte precursor cells as an additional step.

For the purposes of the patent, collection means initially the separation of the melanocyte precursor cells from the first area of the skin. According to the invention, it can also encompass a detachment of the melanocyte precursor cells from the first area of the skin. This detachment can be, for example, simply plucking hairs, in particular anagen hairs from the scalp.

The melanocyte precursor cells applied to the skin of the recipient can thereby in particular also come from this recipient himself. The donor and the recipient can therefore be identical. If this is the case, efforts relating to a typification or matching based on genetic differences between the donor and recipient are thus advantageously omitted or reduced. Furthermore, risks of the transmission, for example, of infection from the donor to the recipient, thus do not apply.

In addition to the separation of the melanocyte precursor cells from the first area—or alternatively thereto—the “collection” of melanocyte precursor cells from root sheaths according to the invention can also include the isolation of the melanocyte precursor cells from the root sheaths, in particular from epithelial root sheaths.

The step of “collection” can also include one step or a plurality of steps by means of which the cells collected are prepared for their application. This can be carried out by means of the production of a melanocyte precursor cell suspension directly or after in vitro multiplication. In addition to the melanocyte precursor cells, this suspension can also comprise biocompatible substances such as PBS and/or fibrin and/or a biocompatible carrier such as, for example, hyaluronan or collagen.

In a further preferred embodiment according to the invention it is proposed that additionally keratinocyte precursor cells are applied. This can take place at the same time as the step of the application of the melanocyte precursor cells. However, the keratinocyte precursor cells and the melanocyte precursor cells can also be applied offset in terms of time. In a further preferred embodiment, the above regarding the collection of the melanocyte precursor cells also applies to the keratinocyte precursor cells. The above regarding the origin of the cells (animal, human, autogenous, autologous, etc.) also applies to the keratinocyte precursor cells.

An advantage that can be achieved by means of the method of these last two embodiments according to the invention lies in that the common and optionally also simultaneous use of melanocyte precursor cells and keratinocyte precursor cells leads to a promoted growth after their common application onto the second area of skin. The inventors of the present method attribute this to interactions of a chemical, biochemical and/or biological nature between the cited precursor cell types. They were able to observe that this advantage already occurs when the natural mixing ratio of keratinocyte precursor cells to melanocyte precursor cells, such as is present, for example, in the root sheaths of the first area of skin, is also maintained in the mixture of these cells applied to the second area of skin.

In a further preferred embodiment of the method according to the invention, the second area of skin is prepared for receiving the melanocyte precursor cells—and optionally also the keratinocyte precursor cells. This preparation permits particularly effectively a growth of the applied precursor cells on the second area of skin.

The preparation of the second area of skin can comprise, for example, a removal of the epidermis, or parts thereof, of the second area of skin. The latter is possible, for example, by means of dermabrasion or superficial laser application with the advantages associated herewith and known to one skilled in the art.

The preparation can also include the application of a suitable solution. As an example thereof a fibrin solution is cited, which prepares the second area of skin for later receiving the precursor cells and for the better adhesion thereof and above all the growth thereof on the second area of skin.

In a further preferred embodiment of the method according to the invention, the precursor cells applied to the second area of skin are stimulated for accelerated development of pigments.

A stimulation of this type can be carried out by means of UV exposure. This activates the transferred melanocyte precursor cells and can be carried out, for example, by means of broadband UV, narrow band UV, PUVA or excimer laser irradiation.

This stimulation advantageously leads to a pigmentation as well as the development of the epidermal pigment unit. A stimulation, for example, by means of ultraviolet light can be carried out once or repeatedly. In each case the irradiation should thereby advantageously be below the threshold erythema dose. An irradiation twice per week until the desired pigmentation of the second area of skin has been achieved has proven to be effective thereby. A more or less frequent irradiation is likewise possible.

The object according to the invention is furthermore attained through the use of melanocyte precursor cells according to the features of claim 7. Advantageous further developments are also hereby in turn the subject matter of respectively the dependent claims.

Since the same advantages can be achieved with the method with the features of claim 7 as with the method described above according to claim 1, to avoid repetitions, at this point the above discussion is explicitly referred to.

The present invention furthermore discloses a method for producing a suspension for use in one of the methods discussed above, wherein advantageous further developments in turn are also hereby the subject of the dependent claims.

It is thus provided with the method according to the invention according to claim 11 to detach the precursor cells—whether they are the melanocyte precursor cells or the keratinocyte precursor cells—enzymatically from the root sheaths of a removed hair. The enzymatic detachment can be carried out, for example, by means of a trypsin/EDTA solution. This solution can be present as 0.8% and have room temperature, for example, 20° C. The solution causes in particular a detachment of the epithelial cells from the hair shaft.

For enzymatic detachment an incubation can take place in trypsin, 0.1% to 10%, in particular between 0.5% to 4%, in PBS over 10-50, in particular over 15-30 mins.

It is in particular advantageous in this approach that a disadvantageous effect on the treated precursor cells, for example, can be avoided by means of the possible use of enzymes. The enzymatic detachment is therefore particularly gentle for the cells to be obtained.

Further possible steps of this method comprise stopping the enzymatic detachment by the addition of human serum, centrifuging the suspension to obtain a sediment as well as the renewed suspension of the cell-containing sediment in a thrombin solution, which permits an immediate fixing of the applied cells in a thin layer in the application on fibrinogen applied beforehand, and thus renders possible a homogeneous, not too occlusive application in any region of the body.

In further preferred embodiments—respectively independently of one another—the method comprises the steps: conversion of the precursor cells or the suspension or the sediment into a biocompatible solution, inclusion of the precursor cells or the suspension or the sediment into a biocompatible carrier and/or production of a cell extract.

An exemplary working example is described in detail below:

In the case of a person with a vitiligo formation of approximately ten square centimeters in area on the back of the hand unchanged for years, 50 anagen hairs were plucked from the scalp. The separated hair roots were incubated for 25 mins in trypsin/EDTA solution, 0.8% at room temperature. Epithelial cells hereby detach from the hair shaft. The reaction was ended by the addition of human serum. The vitality of the cells measured with the trypan blue exclusion was thereby over 50%. The cell suspension was centrifuged at 500 g and the cell-containing sediment was suspended in 2 ml of a thrombin solution. The area of the back of the hand intended for increasing the pigmentation was deepidermized after thorough disinfection by means of dermabrasion. After the application of 2 ml of a fibrin solution on the area of the wound, the cell suspension was applied. The treatment area was subsequently occlusively covered with a conventional wound dressing. The dressing was changed every three days. After a reepithelization completed within two weeks, the treatment area was irradiated twice a week with broad spectrum ultraviolet below the threshold erythema dose. A complete match of the pigmentation of the treated hand area to the skin surrounding it could be achieved in this manner in the course of eight weeks.

To suppress autoimmune reactions, in the case of a vitiligo suitable active ingredients such as topical or systemic corticosteroids or topical calcineurin inhibitors can be used for a limited time.

The present invention proposes a cosmetic method for increasing the pigmentation of the skin with the steps application of melanocyte precursor cells from root sheaths, which were collected from a first area of skin of a donor to a second area of skin of a recipient. Furthermore, the use of melanocyte precursor cells for increasing the pigmentation of skin as well as a method for producing a suspension with precursor cells from the root sheath is proposed.

1.-17. (canceled)

18. A cosmetic method for increasing the pigmentation of skin, wherein the method comprises applying melanocyte precursor cells from hair root sheaths collected from an area of donor skin to an area of recipient skin whose pigmentation is to be increased.

19. The method of claim 18, wherein donor and recipient are identical.

20. The method of claim 18, wherein the method further comprises preparing the collected melanocyte precursor cells prior to applying them to the area of recipient skin.

21. The method of claim 18, wherein the method further comprises applying keratinocyte precursor cells to the area of recipient skin.

22. The method of claim 18, wherein the method further comprises preparing the area of recipient skin for receiving precursor cells.

23. The method of claim 18, wherein the method further comprises stimulating precursor cells to develop pigments following the application of the precursor cells to the area of recipient skin.

24. The method of claim 18, wherein the donor is a human.

25. The method of claim 18, wherein the recipient is a human.

26. A method of using melanocyte precursor cells from hair root sheaths for increasing the pigmentation of a skin area, wherein the method comprises collecting the precursor cells from an area of donor skin and applying the collected precursor cells to an area of recipient skin whose pigmentation is to be increased.

27. The method of claim 26, wherein donor and recipient are identical.

28. The method of claim 26, wherein keratinocyte precursor cells are additionally employed.

29. A method for producing a suspension of precursor cells for use in the method of claim 18, wherein the method comprises an enzymatic detachment of the precursor cells from the hair root sheaths and suspending them in a liquid.

30. The method of claim 29, wherein the method further comprises stopping the enzymatic detachment by adding human serum.

31. The method of claim 29, wherein the method further comprises subjecting the suspension to a centrifugation to obtain a cell-containing sediment.

32. The method of claim 31, wherein the method further comprises suspending the sediment in a thrombin solution.

33. The method of claim 29, wherein the method further comprises converting the suspension of precursor cells into a biocompatible solution.

34. The method of claim 31, wherein the method further comprises converting the sediment into a biocompatible solution.

35. The method of claim 29, wherein the method further comprises including the precursor cells in a biocompatible carrier.

36. The method of claim 31, wherein the method further comprises including the sediment in a biocompatible carrier.

37. The method of claim 29, wherein the method further comprises preparing a cell extract.
